(19) United States
(12) Patent Application Publication
(10) Pub. No.: US 2021/0183500 A1
(43) Pub. Date: Jun. 17, 2021

(54) Smartphone Application for Medical Image Data Sharing and Team Activation

(71) Applicant: Duke University, Durham, NC (US)

(72) Inventors: Aditya Mandawat, Durham, NC (US); Alexander Blood, Durham, NC (US); Sunil Rao, Durham, NC (US); Schuyler Jones, Durham, NC (US); Manesh Patel, Durham, NC (US)

(21) Appl. No.: 16/636,524

(22) PCT Filed: Aug. 6, 2018
(86) PCT No.: PCT/US2018/045352

§ 371 Date: Feb. 4, 2020

Related U.S. Application Data
(60) Provisional application No. 62/541,869, filed on Aug. 7, 2017.
(60) Provisional application No. 62/670,576, filed on May 11, 2018.

(51) Int. Cl. G16H 40/20; G16H 30/20; G16H 30/40; G16H 40/67; G16H 10/60; A61B 6/00; G01S 19/42

(52) U.S. Cl. CPC G16H 40/20; G16H 30/20; G16H 30/40; G06F 3/04847; G16H 10/60; A61B 6/56; G01S 19/42; G16H 40/67

(57) ABSTRACT

A method includes receiving medical image data from an image sharing application running on a sharing device. The method further includes determining a category of medical information represented in the medical image data. The method also includes identifying a viewing device to view the medical image data. The method additionally includes transmitting, to the viewing device, a request for confirmation of a medical condition associated with the category of medical information, wherein the request allows the viewing device to view the medical image data in an image viewing application running on the viewing device before an end of a timeout period. The method further includes receiving, from the viewing device, a signal confirming the medical condition based on the medical image data. The method also includes in response to receiving the signal, transmitting an activation signal to each member user device of a medical team associated with the viewing device.

### CROSS-REFERENCE TO RELATED APPLICATIONS

This application claims priority to U.S. Provisional Patent Application No. 62/541,869, filed Aug. 7, 2017, and U.S. Provisional Patent Application No. 62/670,576, filed May 11, 2018, each of which are incorporated herein by reference.

### BACKGROUND

One of the major challenges that healthcare providers face is communication. This challenge is due in part to legal issues around the handling and transfer of medical data, including the Health Insurance Portability and Accountability Act (HIPAA). This challenge is also due to the absence of a uniform system of medical records. The inability to deliver relevant, accurate, and timely information to healthcare providers may sometimes lead to inefficient medical triage, unnecessary wait times, missing information during medical care, and worse outcomes for patients.

A ST-Elevation Myocardial Infarction (STEMI) is the most serious type of heart attack, during which one or more of the heart's arteries are completely blocked. As such, medical guidelines for the management of STEM mandate primary percutaneous coronary intervention (PCI) within a short time window (e.g., within 90 minutes of first medical contact). Such requirements may put pressure on healthcare systems to activate a cardiac catheterization laboratory (or cath lab) before a full review of a patient's presenting condition may be completed. This may lead to false cath lab activation in cases where a true STEMI is not present. False cath lab activations result in increased costs, decreased cath lab availability for true myocardial infarctions, and decreased staff resiliency and morale. Accordingly, systems that help to reduce false cath lab activations may be desirable.

### SUMMARY

In an aspect, a method is provided. The method includes receiving medical image data from an image sharing application running on a sharing device. The method further includes determining a category of medical information represented in the medical image data. The method also includes identifying, based on the category of medical information, a viewing device to view the medical image data. The method additionally includes transmitting, to the viewing device, a request for confirmation of a medical condition associated with the category of medical information, wherein the request allows the viewing device to view the medical image data in an image viewing application running on the viewing device before an end of a timeout period. The method further includes receiving, from the viewing device, a signal confirming the medical condition based on the medical image data. The method also includes in response to receiving the signal, transmitting an activation signal to each member user device of a medical team associated with the viewing device, wherein the activation signal requires closed-loop acknowledgement by each member user device that activation has occurred.

In another aspect, a non-transitory computer readable medium is provided having stored therein instructions executable by one or more processors to cause the one or more processors to perform functions. The functions include receiving medical image data from an image sharing application running on a sharing device. The functions further include determining a category of medical information represented in the medical image data. The functions also include identifying, based on the category of medical information, a viewing device to view the medical image data. The functions additionally include transmitting, to the viewing device, a request for confirmation of a medical condition associated with the category of medical information, wherein the request allows the viewing device to view the medical image data in an image viewing application running on the viewing device before an end of a timeout period. The functions further include receiving, from the viewing device, a signal confirming the medical condition based on the medical image data. The functions also include in response to receiving the signal, transmitting an activation signal to each member user device of a medical team associated with the viewing device, wherein the activation signal requires closed-loop acknowledgement by each member user device that activation has occurred.

In a further aspect, a system is provided comprising one or more processors and a non-transitory computer readable medium having stored therein instructions executable by the one or more processors to cause the one or more processors to perform functions. The functions include receiving medical image data from an image sharing application running on a sharing device. The functions further include determining a category of medical information represented in the medical image data. The functions also include identifying, based on the category of medical information, a viewing device to view the medical image data. The functions additionally include transmitting, to the viewing device, a request for confirmation of a medical condition associated with the category of medical information, wherein the request allows the viewing device to view the medical image data in an image viewing application running on the viewing device before an end of a timeout period. The functions further include receiving, from the viewing device, a signal confirming the medical condition based on the medical image data. The functions also include in response to receiving the signal, transmitting an activation signal to each member user device of a medical team associated with the viewing device, wherein the activation signal requires closed-loop acknowledgement by each member user device that activation has occurred.

In yet a further aspect, a system is provided that includes means for receiving medical image data from an image sharing application running on a sharing device. The system further includes means for determining a category of medical information represented in the medical image data. The system also includes means for identifying, based on the category of medical information, a viewing device to view the medical image data. The system additionally includes means for transmitting, to the viewing device, a request for confirmation of a medical condition associated with the category of medical information, wherein the request allows the viewing device to view the medical image data in an image viewing application running on the viewing device before an end of a timeout period. The system further includes means for receiving, from the viewing device, a signal confirming the medical condition based on the medical image data. The system also includes means for in response to receiving the signal, transmitting an activation signal to each member user device of a medical team associated with the viewing device, wherein the activation signal requires closed-loop acknowledgement by each member user device that activation has occurred.

In an additional aspect, a method is provided. The method includes capturing an electrocardiogram (ECGs or EKG) image with a camera of a mobile device from an application running on the mobile device. The method further includes identifying a destination hospital with a recipient for the EKG image. The method additionally includes determining an estimated time of arrival (ETA) to the destination hospital. The method also includes generating, by the application, a digital EKG object comprising the EKG image and associated metadata, where the associated metadata includes the recipient and the ETA. The method further includes transmitting the digital EKG object from the mobile device to a remote server, where the EKG image and the ETA are viewable for a limited time duration by the recipient from a viewing device in communication with the remote server.

In another aspect, a mobile device is provided that is configured to capture an electrocardiogram (EKG) image with a camera of the mobile device from an application running on the mobile device. The mobile device is further configured to identify a destination hospital with a recipient for the EKG image. The mobile device is additionally configured to determine an estimated time of arrival (ETA) to the destination hospital. The mobile device is further configured to generate, by the application, a digital EKG object comprising the EKG image and associated metadata, where the associated metadata includes the recipient and the ETA. The mobile device is also configured to transmit the digital EKG object from the mobile device to a remote server, where the EKG image and the ETA are viewable for a limited time duration by the recipient from a viewing device in communication with the remote server.

In a further aspect, a non-transitory computer readable medium is provided having stored therein instructions executable by one or more processors to cause a mobile device to perform functions. The functions include capturing an electrocardiogram (EKG) image with a camera of the mobile device from an application running on the mobile device. The functions also include identifying a destination hospital with a recipient for the EKG image. The functions additionally include determining an estimated time of arrival (ETA) to the destination hospital. The functions further include generating, by the application, a digital EKG object comprising the EKG image and associated metadata, where the associated metadata includes the recipient and the ETA. The functions additionally include transmitting the digital EKG object from the mobile device to a remote server, where the EKG image and the ETA are viewable for a limited time duration by the recipient from a viewing device in communication with the remote server.

In yet a further aspect, a system is provided that includes means for capturing an electrocardiogram (EKG) image. The system also includes means for identifying a destination hospital with a recipient for the EKG image. The system additionally includes means for determining an estimated time of arrival (ETA) to the destination hospital. The system further includes means for generating a digital EKG object comprising the EKG image and associated metadata, where the associated metadata includes the recipient and the ETA. The system additionally includes means for transmitting the digital EKG object to a remote server, where the EKG image and the ETA are viewable for a limited time duration by the recipient from a viewing device in communication with the remote server.

These as well as other embodiments, aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE FIGURES

FIG. 1 illustrates communication between a sharing device and a server, in accordance with example embodiments.

FIG. 2 illustrates communication between a server and a viewing device, in accordance with example embodiments.

FIG. 3 illustrates additional communication between a server and a viewing device, in accordance with example embodiments.

FIG. 4 illustrates communication between a server and member user devices of a medical team, in accordance with example embodiments.

FIG. 5 illustrates additional communication between a server and member user devices of a medical team, in accordance with example embodiments.

FIG. 6 is a block diagram of a method, in accordance with example embodiments.

FIG. 7 is a data flow diagram, in accordance with example embodiments.

FIGS. 8A-8G illustrate application screenshots on a sender device, in accordance with example embodiments.

FIG. 9 illustrates an application screenshot on a viewer device, in accordance with example embodiments.

FIG. 10 is a block diagram of a method, in accordance with example embodiments.

FIG. 11 is a schematic illustrating a conceptual partial view of an example computer program product that includes a computer program for executing a computer process on a computing device, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features. Other embodiments can be utilized, and other changes can be made, without departing from the scope of the subject matter presented herein.

Thus, the example embodiments described herein are not meant to be limiting. Aspects of the present disclosure, as generally described herein, and illustrated in the figures, can be arranged, substituted, combined, separated, and designed in a wide variety of different configurations, all of which are contemplated herein.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Example embodiments involve transient sharing of medical image data for purposes of efficient patient evaluation and treatment. Such embodiments have applications in a number of settings. One such setting is emergency medical services (EMS) triage. In the evaluation of a patient, EMS often assists healthcare providers at a hospital by providing information while in route to the hospital. While this information sharing is routinely completed via a radio or phone call, EMS providers are often engaged in multiple mediums of documentation during their care of a patient. This information rarely is made available to hospital providers who rely on verbal handoff and on the reassessment of hospital staff to plan and execute treatment decisions. Especially in acute conditions that need rapid medical evaluation and treatment, EMS teams often activate hospital resources while in transit in order to minimize delays in providing care. These activations sometimes occur without providing information necessary for the care of a patient to those tasked with caring for the patient.

Example embodiments also have applications in the area of hospital transfer. When transferring a patient from a clinic or urgent care to an emergency room or from a hospital to a hospital at a higher level of care, a legal requirement exists under the Emergency Medical Treatment and Labor Act (EMTALA) to provide information and documentation relevant to the patient's care to the receiving institution. While egregious infractions may be reported, patients are often sent without complete or coherent medical information (including imaging) that is difficult, expensive, and perhaps even dangerous to repeat. This makes caring for the patient slower and more difficult as delays in acquiring the data accumulate.

Example embodiments additionally have applications in the area of emergency department consultations. When evaluating a patient in the emergency room, physicians there often rely on the expertise from colleagues in diverse disciplines including Cardiology, Ophthalmology, Gynecology, Cardiac Surgery, Neurology, Orthopedic Surgery, and Neurosurgery; these providers are often not physically present when their knowledge and experience is called for. Consultations from expert physicians from the emergency department may require physicians to change their schedule, travel from off-site and present at all hours of the night to determine how to best triage and care for these patients. Accordingly, consultation requests with medical image data may be routed to a centralized location for an external case review. For instance, a remotely located cardiologist may receive case review requests from emergency room doctors at a number of different facilities, including smaller hospitals that may not have an on-site cardiologist immediately available. More generally, an example system may allow a number of different types of medical providers (e.g., emergency room doctors, primary care doctors, urgent care doctors, or nurse practitioners) to request patient triage or management advice from a number of different types of remote specialists (e.g., dermatologists, cardiologists, ophthalmologists, or pathologists).

In the setting of transitions of care in between providers, care settings, and institutions, patients often do not have access to their own medical data that may be important in the evaluation and management of their medical conditions. Because of the threat of loss of life or limb, clinicians often opt to repeat expensive laboratory tests, imaging studies, and even potentially risky invasive diagnostic procedures to determine the best course of evaluation and treatment. These inefficiencies lead to substantial costs.

Example embodiments described herein involve a software application running on the smartphones (or other portable user devices) of medical professionals to allow for the transient sharing of medical image data without storing the image data to the smartphones. Example use cases include EMS triage to evaluate patients, transfer of patients between hospitals, and consultations to evaluate patients in the emergency room. Backend and frontend functionality may be provided to facilitate the transfer of clinically relevant medical information in an efficient and secure (e.g., HIPAA-compliant) manner. Additionally, an image sharing application may close the loop to ensure that a clinical decision is made for a patient by an appropriate decision maker and that an appropriate team of providers is activated if needed to care for the patient.

Example types of medical image data that may be shared include magnetic resonance imaging (MRI) scans, electrocardiogram (EKG or ECG) printouts, X-rays, coronary angiograms, fundoscopic scans, and computed tomography (CT) scans. Additional types of medical image data that may be shared include patient images, such as skin, eye, or tissue images. In further examples, shared medical image data may also include video and/or audio data. In the case of video, a user of a sharing device may select which frames of video data to share. Video data captured by a smartphone camera may be encoded in manner that allows a user to scroll through the video to select clinically relevant frames for sharing. In further examples, audio data (e.g., murmurs from a potential stroke patient) may be sent in addition to or instead of video data.

On the backend, some example systems may be designed to satisfy an overall goal of minimizing the amount of time that medical image data is available while solving a clinical goal. In particular, the medical image data may be erased at the end of a timeout window, and the timeout window for transient image data may be adjusted based on the clinical use case. The timeout window may be adjusted based on the category of information represented in the medical image data. For instance, information needed for rounding may be stored for 24 hours or 48 hours while other information such as EKGs may only be stored until a patient is expected to arrive at a hospital. The timeout window may be adjusted based on an estimated time of arrival (ETA) of a sharing device to a hospital, the ETA of a viewing device to a hospital, or both. Global Positioning System (GPS) data may be used to estimate ETAs for the sharing device and/or the viewing device. The ETA of a patient to a hospital may also be stored and made available to the viewing device. The timeout window for different use cases may be set by a hospital or administrative backend. The timeout window may also be based on user input data entered at the sharing device, the viewing device, or both. A handshake process may allow the users of the sharing device and the viewing device to mutually agree on a timeout window. A proposed timeout window may also be autonomously generated, and users may then be allowed to adjust the proposed timeout window.

While many use cases benefit from making the data transient, it may be desirable to permanently store some image data instead (e.g., for machine learning applications, for clinical reasons, and/or for legal purposes). A toggleable option (e.g., a slide bar) may allow a user of a sharing device to switch between making subsequently captured images transient and permanently saving image data. Permanently stored image data in a database may be linked to a patient so that the data can be referenced later.

In further examples, backend routing functionality may be provided as part of an image sharing application to route medical images to the devices of appropriate medical professionals. A major challenge faced by medical providers at the regional level is identifying the right doctor or group of doctors that needs to be consulted in a fast and efficient manner. When image data for a patient comes in through the application, the image data may be analyzed on the backend to determine a doctor or group of doctors to receive the image data. In some cases, this routing process may involve categorizing the image data into one of a number of predefined categories of medical information. Image data may be categorized by the type of medical imaging technology represented in the image data (e.g., MRI, EKG, X-ray, CT scan). Image data may also be categorized by the type of medical condition or the type of clinical decision needed. For instance, example categories could include acute myocardial infarction, pulmonary embolism, aortic dissection, thoracic dissection, stroke, and shock. The platform may be provided with information about respective responsibilities of individual physicians and groups of physicians in order to appropriately route incoming image data.

In additional examples, scheduling functionality may also be provided to ensure that available doctors or other medical professionals receive needed information to make a clinical decision and confirm via closed-loop communication that they are activated to provide medical services when needed. Hospitals often spend a significant amount of resources on pagers to ensure physicians have received critical messages about patients. In some examples, a medical image sharing application may provide a full proof alternative to pagers. In particular, medical image data may be shared with a viewing device along with a request for confirmation of a medical condition associated with the type of medical image data shared. For instance, an EKG printout may be shared along with a request to confirm the presence of a ST-Elevation Myocardial Infarction (STEMI). The application running on the viewing device may provide a prompt to ensure that the viewer has received the image data, can interpret the image data, and has made a relevant clinical decision. A confirmation user input signal inputted by a user of a viewing device may therefore serve a similar function as a doctor making a confirmation phone call after receiving a pager message about a patient.

A scheduling system may be used to identify an “on-call” provider at a facility who is automatically routed medical image data for purposes of evaluation, triage, and management. In particular, an example scheduling system may identify an on-call decision maker for a given type of medical image data. The decision maker may first receive a request to confirm that the patient has a particular medical condition. The decision maker may be able to view shared image data for the patient for a limited time window to facilitate a diagnosis. If the decision maker confirms that a particular medical condition is present (e.g., by pressing a digital button in the application), the scheduling system may then identify and send activation signals to each member user device of a medical team associated with the decision maker for the particular medical condition. The activation signals may require closed-loop acknowledgement by each team member to confirm that they have received the message and will perform their respective tasks to prepare to treat a patient (e.g., prepare a cath lab for a STEMI). In this manner, a medical image sharing application may close the loop to ensure that a medical decision has been made by an appropriate physician and that an appropriate team of medical professionals will be ready to treat the patient if necessary. In further examples, application alerts, phone calls, and/or paging may be incorporated in order to confirm a response from each member of an on-call medical team. In additional examples, activation signals sent to each member user device may also activate relevant mobile device functionality, such as GPS tracking.

On the frontend, software functionality may be provided to assist a user of a sharing device to share clinically relevant information. Confidential information, such as personal health information (PHI), may be blocked out. Bounding boxes and/or cropping may be used to allow a user to customize images before sharing. Image processing may be performed to facilitate customization of shared images. Overlaid boxes that black out certain areas of an image (e.g., name, birthday) may be suggested to a user. The suggested portions of an image to share or block out may be subject to user override. Image processing may also be performed for validation. For instance, an image may be identified as an MRI and processed to make sure it is a valid and interpretable MRI image. The identity of a patient may also be verified.

A user of the viewing device may also be asked to confirm that medical image data is interpretable. For instance, the user can give an electronic signature or thumbprint to confirm that the user has received the image and can make a clinical decision based on the image. If not, a request may be sent to the sharing device to provide more medical image data.

In further examples, a collection of medical image data may be compiled to create a digital rounding sheet that will be made available digitally to doctors doing rounds at a hospital. This digital document may include relevant image data (e.g., X-rays, CT scans, MRIs), lab values, pertinent notes, and discharge summaries. Patient release and signature functionality may also be built into the application to allow for the transfer of medical records.

Additional example systems may use machine learning techniques to train a medical image sharing application to perform image recognition to facilitate better image sharing and/or clinical decision making. At a first level, machine learning may be used to ensure that particular types of medical image data (e.g., CT scans, X-rays) will be interpretable by a user of a viewing device. For instance, a machine learning model may be trained based on bounding boxes selected by users of sharing devices in order to learn how to automatically size a bounding box on the sharing device. Feedback from users of viewing devices (e.g., whether they request additional medical image data before making a clinical decision) may also be used to train a model to assist with the interpretability problem.

At a second level, machine learning may also be used to train a model to automatically make clinical decisions as well or instead. For instance, the model may be trained by cardiologist decisions for different EKG images. By storing these cardiologist decisions and corresponding EKG images in a database, the model may be trained to learn how to use image recognition to make clinical decisions that would align with those made by a human doctor. Over time, the model may reach a confidence level where the model can automatically make a decision to activate or not active a cath lab without additional input from a separate user of a viewing device. In further examples, a machine learning model may be trained based on other types of medical image data to automatically make other types of clinical decisions as well.

Some example systems and methods involve EKGs, which are critical in the diagnosis of STEMIs. Pre-hospital transmissions of EKGs by emergency medical services (EMS) allow hospital doctors to evaluate a patient's EKG and make an informed decision about cath lab activation. Upgrading EMS and hospital systems with equipment to transmit EKGs from the field is currently cost-prohibitive for many hospitals. In addition, available transmission systems are proprietary and tied to a specific company's devices, precluding a simple, low cost solution that may be adopted by all hospital and EMS systems.

In the era of camera phones, a digital picture of the pre-hospital EKG may be obtained and transmitted via short message service (SMS) or email to the hospital. However, such systems raise concerns around privacy, regulatory compliance, and reliability. Example embodiments described herein include a mobile device application (e.g., a smartphone application) that permits the pre-hospital transmission of EKGs from the field. In examples, the mobile device application may be designed to be compliant with the Health Insurance Portability and Accountability Act (HIPAA). Example features of the application may include encryption and time-limited storage of EKGs.

More specifically, the application may include a number of features to facilitate interaction between EMS in the field and a cardiologist at a hospital. In particular, the application may include the ability to take high-resolution photos of the EKG. Such photos may reside transiently within a secure space within the application and not on the smartphone, and may be permanently deleted from the smartphone immediately following transmission. The application may additionally include the ability to transmit such photos securely to the mobile phone of a cardiology provider. All communication to and from the server may be secured by Hyper Text Transfer Protocol Secure (HTTPS). The application may also include the ability for EMS to confirm the successful transmission of the EKG, via voice contact with providers at the hospital and/or automatically via quality assurance routines within the application. The application may further include the ability for hospital providers to contact EMS providers who send the EKG from within the application. The application may also include a real-time estimated time of arrival of EMS to the hospital, represented by a fixed or updating number and/or on a map. The application may additionally include the ability to permanently delete photos of the EKG from the server after a predetermined time period (e.g., 120 minutes). Of note, this does not constitute destruction of medical information as the original paper EKG would still be brought by EMS to the hospital. The application may additionally include the ability to record de-identified metadata and transmit such data to a secure database.

A smartphone application with some combination of such features may enable EMS systems to securely transmit EKGs (e.g., standard 12-lead EKGs) from the field to hospital doctors to allow for more rational activation of the cardiac catheterization laboratory. Additional features that may be included in example systems are described below with reference to the accompanying figures.

Additional features that may be included in example systems are described below with reference to the accompanying figures.

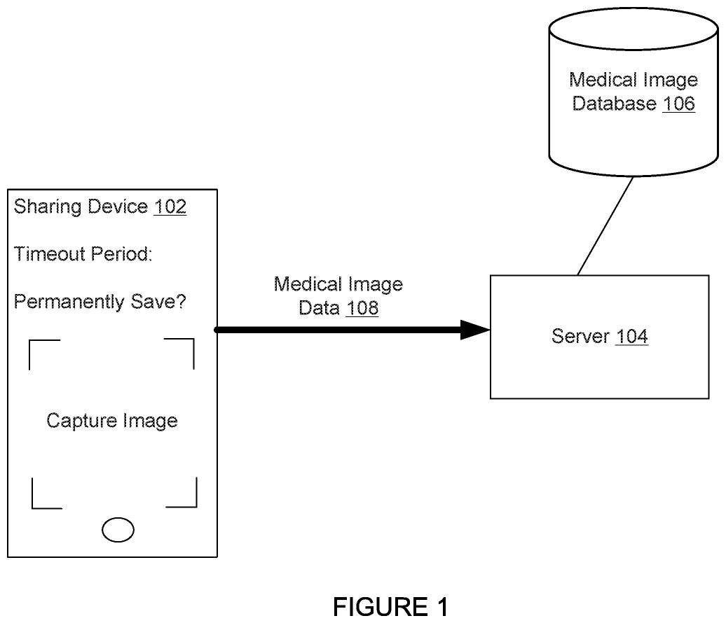

FIG. 1 illustrates communication between a sharing device and a server, in accordance with example embodiments. More specifically, an image sharing software application running on sharing device 102 may allow for the capture and transfer of medical image data 108 to server 104. Sharing device 102 may be a smartphone or a different type of mobile or fixed computing device. Sharing device 102 may transmit medical image data 108 to server 104 via a wireless connection or a different type of connection. The server 104 may store medical image data 108 in medical image database 106 in order to share medical image data 108 with one or more viewing devices. To protect confidential patient medical information, medical image data 108 may be erased by server 104 from medical image database 106 at the end of a timeout period. Additionally, medical image data 108 may not be stored to the sharing device 102 by the software application running on sharing device 102. Accordingly, medical image data 108 may be inaccessible by sharing device 102 after the medical image data 108 is transmitted to server 104.

The software application running on sharing device 102 may include user interface (UI) functionality to facilitate the sharing of clinically relevant snapshots making up medical image data 108. The UI functionality may include an adjustable bounding box set by a user of sharing device 102 in order to exclude unnecessary PHI or other information. The UI functionality may also include the ability to separately block out regions of a captured image to avoid sharing those regions with a viewing device as well or instead of a bounding box. An indication of the portions of the medical image data 108 to include or exclude when sharing the image data may be stored by server 104 in medical image database 106.

In some examples, image processing may be performed by the software application running on sharing device 102 and/or server 104 in order to determine how to size a bounding box and/or which regions of an image to block out. The software application may then provide suggestions of the bounding box size and/or locations of blocked out regions, which may then be adjusted by a user of sharing device 102. In order to make these suggestions, the software application may determine a type of medical information (e.g., MRI, X-ray) captured in medical image data 108. The software application may then reference a previously stored template with suggested bounding box and/or blocked out region placement for the determined type of medical information. For instance, a bounding box may automatically be set that captures the medically relevant data (e.g., voltage measurements from an EKG printout) while excluding surrounding PHI such as a patient's medical record number, name, and birthday. In other examples, the software application may search for particular pieces of PHI and block out those values in captured medical image data.

In further examples, a machine learning model may be used to help identify which portions of medical image data to share and which portions to block out. The model may be trained over time with user input data setting the bounding box and/or blocked out regions for different types of medical image data. The model may make suggestions which can be edited a user of sharing device 102. In further examples, if the model has a sufficiently high confidence level, captured image data may automatically be processed to determine which regions to include or exclude without further user input.

In additional examples, UI functionality may also allow a user of sharing device 102 to provide input data to select particular frames from video data captured by a video camera of sharing device 102. The video data may be encoded in a manner that allows the user to scroll through the video data to select the most relevant frames (e.g., of an MRI or CT scan) to share in medical image data 108. A user of a viewing device may then be able to scroll through the selected frames rather than playing and pausing the video to make a diagnosis. In further examples, a machine learning model may also be trained to help a user of the sharing device 102 select clinically relevant frames of video data (e.g., by displaying suggested frames to share or automatically processing video data to pick out particular frames for sharing with a viewing device).

In further examples, the software application running on sharing device 102 and/or server 104 may perform one or more validation functions on captured image data before allowing the image data to be shared as medical image data 108. For instance, a validation function may involve validating that the image is one of a predefined set of medical image types that may be shared through the application (e.g., X-rays, MRIs, CT Scans, etc.). An additional validation function may involve verifying that the captured medical image data of the identified type includes interpretable data (e.g., verifying that a captured EKG printout includes interpretable voltage data). A further validation function may involve verifying that information identifying the patient matches expected patient information (e.g., name or birth date).

In some examples, additional UI functionality may allow for the compilation of a digital document associated with a patient. The digital document may include a compilation of different types of medical image data (e.g., pictures of lab values, pictures of vitals) as well as other types of information (e.g., technician notes, a discharge summary as part of a hospital transfer). The digital document may then be accessed by other medical professionals, for instance, as a digital rounding sheet. By compiling information captured on an ambulance or by a transferring hospital, future doctors seeing the same patient may not have to redo costly tests or imaging.

As previously noted, the storage of medical image data may be made transient for confidentiality reasons. However, in some examples, it may be advantageous to adjust the length of time that individual images are stored, or in some cases to permanently store certain images. In particular, this time limit may be set differently based on clinical use case. For instance, an EKG image for a patient may only need to be stored for 45 minutes if the ETA of a patient to the hospital is 40 minutes (based on GPS data from the sharing device 102). By contrast, X-ray data for a patient being driven 12 hours from one hospital to another for a lung transplant may need to be stored for the entire 12-hour drive. In another example, if the medical image data is needed for rounding, it may need to be stored 48 hours or 72 hours. Other types of medical image data may need to be permanently stored for medical record purposes or other reasons.

In some examples, the length of the timeout window and/or whether to permanently store medical image data may be set by a user of sharing device 102. For instance, UI functionality may be provided to allow the user to type in an amount of time to save the data or to turn on permanent saving for a particular image. In other examples, the server 104 may suggested the amount of time and/or whether or not to permanently save the image data, and the user of the sharing device 102 may then make adjustments. In yet other examples, the server 104 may automatically determine how long to save medical image data and/or whether or not to permanently save medical image data without further user input. In some examples, this determination may be made based on an identified type of medical information contained within the medical image data. For instance, the length of time to save an MRI or an X-ray may be predetermined (e.g., set by a particular hospital or administrative backend).

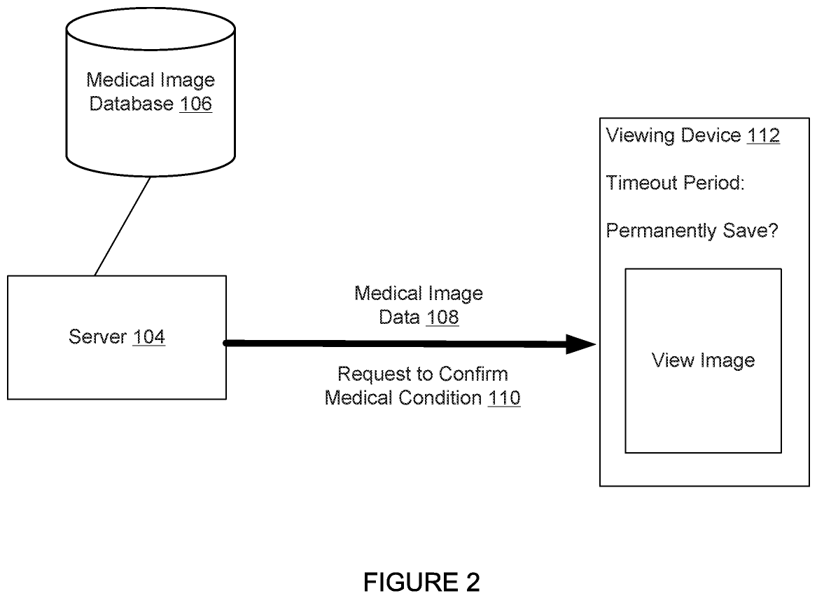

Next, FIG. 2 illustrates communication between a server and a viewing device, in accordance with example embodiments. More specifically, server 104 may make medical image data 108 available for viewing by an image viewing device 112 for a limited time period before erasing medical image data 108 from medical image database 106. Notably, the medical image data 108 may only be viewable within a software application running on the viewing device 112, and may never be stored to the viewing device 112. The viewing device 112 may be a smartphone or a different type of computing device used by a medical professional.

Before making medical image data 108 available for viewing, server 104 may first identify viewing device 112. To identify viewing device 112, server 104 may locate a doctor who is on-call and qualified to handle diagnostic decisions associated with the type of imaging contained in medical image data 108. In some examples, the sharing device 102 may identify a hospital to which a patient will be brought, and the viewing device 112 may be identified by the server 104 from among possible users at the chosen hospital. For instance, an EKG image may be routed to an on-call cardiologist at the target hospital to make a decision about whether or not a STEMI is present. In other examples, the server 104 may consider multiple nearby hospitals in order to locate an appropriate viewing device 112.

A big challenge exists at the regional level in identifying the right doctor that needs to be contacted in an emergency in the fastest way possible. An image sharing platform with many different physicians tied in to the system may allow for the intelligent routing of many different categories of medical images to different providers or sets of providers. Image data may be categorized by the type of medical imaging technology used to generate the images or by the type of clinical decision needed. In some examples, hospitals may be required to use a scheduling application to specify on-call doctors to handle respective categories of medical image data. The server 104 may then reference the current schedule at a particular hospital or multiple hospitals in an area in order to locate an appropriate viewing device 112 for the medical image data 108.

In order to close the loop and ensure that a clinical decision is made for the patient based on the captured medical image data, the server 104 may transmit a request to confirm a medical condition 110. More specifically, the medical condition may be associated with the medical image data 108 (e.g., a STEMI for an EKG image or an aortic dissection for a CT scan). The request 110 may be determined by the server 104 based on the medical image data 108 or the request 110 may be specified by the sending device 102. In some examples, the request 110 may require a “yes” or “no” response from a user of the viewing device 112. In other examples, the request 110 may allow for more than two possible responses (e.g., when multiple different medical conditions can be identified from the medical image data).

In some examples, the server 104 may first send a notification to the viewing device 112 indicating that there is a pending request 110 and available medical image data 108. The notification may appear in a pending notification list on viewing device 112. The notification may include an amount of time by which the viewing device 112 is expected to respond. For instance, this amount of time may be based on an ETA of the sharing device 102 to the hospital. If the user of the viewing device 112 does not respond in a certain amount of time, a reminder may be sent to the viewing device 112 to get the user's attention. In further examples, additional viewing devices may be contacted in order to ensure that a response to the request 110 is received.

A user of the viewing device 112 may only be able to view the medical image data 108 and the request 110 within an image viewing software application running on viewing device 112. Accordingly, the patient's medical information may never be stored to the viewing device 112. Additionally, at the end of the timeout period, the viewing device 112 may no longer be able to access the medical image data 108 for the patient.

In some examples, the user of the viewing device 112 may be able to specify or contribute to the determination of the timeout period for the medical image data 108 and/or whether or not to permanently save the medical image data 108. For instance, the viewing device 112 may display the planned length of time to save the medical image data 108, which may have been autonomously determined by server 104 and/or specified by sharing device 102. The user of the viewing device 112 may then be able to modify the timeout period. In some examples, the server 104 may facilitate a handshake process in which the user of both the sharing device 102 and the viewing device 112 must agree on the length of the timeout period and/or whether or not to permanently save the medical image data 108. The server 104 may also provide an indication of the timeout period on the sharing device 102 and/or the viewing device 112 to allow for adjustments.

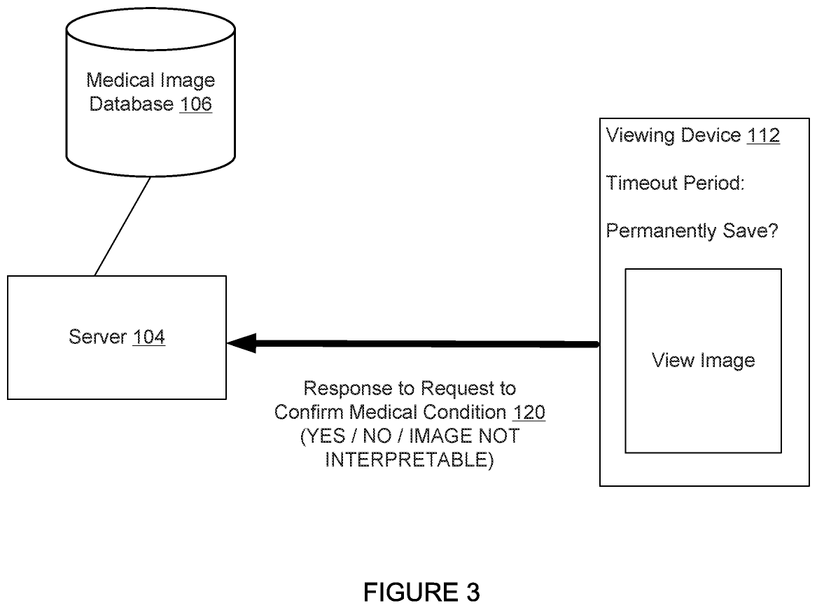

Next, FIG. 3 illustrates additional communication between a server and a viewing device, in accordance with example embodiments. More specifically, after viewing medical image data 108 on viewing device 112, a user of viewing device 112 may provide input data that triggers the transmission of a message to server 104. The message may be a response 120 to the request 110 to confirm a medical condition. For instance, a doctor using viewing device 112 may confirm the presence of a STEMI after viewing an EKG printout on viewing device 112. The image viewing software application running on viewing device 112 may therefore ensure that a relevant clinical decision has been made for the patient based on the shared medical image data 108.

In some examples, server 104 may transmit a request to confirm that the medical image data 108 is interpretable in addition to or instead of the request 110 to confirm the presence of a medical condition. If the user of the viewing device 112 provides a response indicating that the medical image data 108 is not sufficiently interpretable to make a relevant clinical decision, the server 104 may be configured to responsively transmit a message to the sharing device 102 to request additional image data from the sharing device 102. After receiving additional image data from sharing device 102, server 104 may transmit another request to the viewing device 112 to ensure that the image data is interpretable and/or to request confirmation of a medical condition. In this manner, the server 104 may close the loop to ensure that a shared medical image has been received, has been viewed, and is sufficiently interpretable by the receiving doctor to make a clinical decision for the patient.

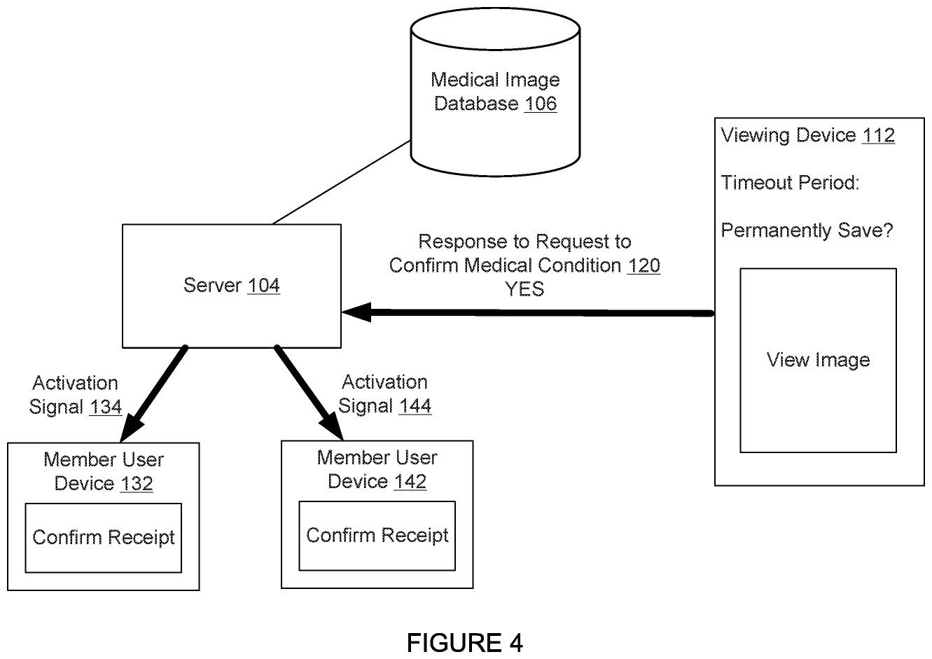

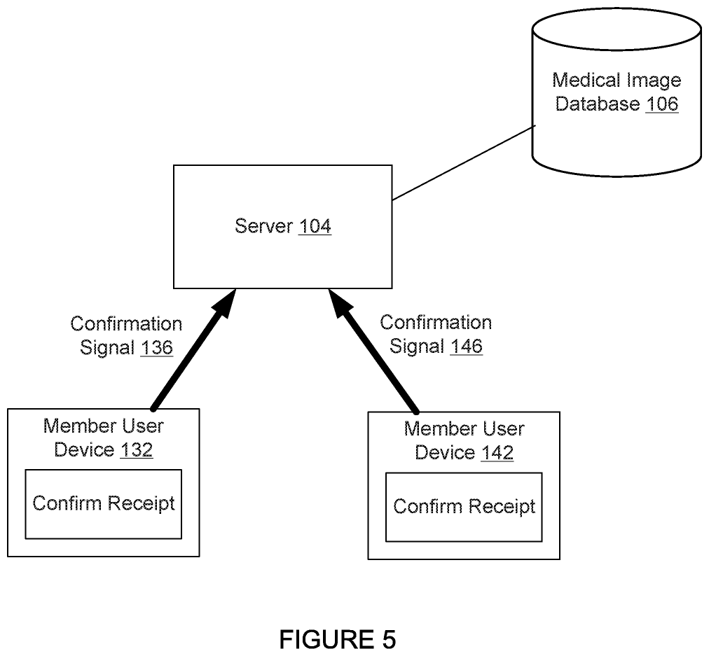

In some examples, the response 120 may indicate that a medical condition such as a STEMI is not present. The server 104 may take no further action at that point or it may send another message to the sharing device 102 to relay the response 120. In other examples, the response 120 may indicate that a medical condition is present, and the server 104 may respond only by transmitting the response 120 to the sharing device 102 (such as when the user of sharing device 102 requests input from an off-site consulting doctor using viewing device 112). In further examples, upon receiving a response 120 indicating that a medical condition is present, server 104 may take additional steps to ensure that an appropriate team of medical providers is activated to prepare to treat the patient, as illustrated in FIGS. 4 and 5.

Next, FIG. 4 illustrates communication between a server and each member user device of a medical team, in accordance with example embodiments. More specifically, the viewing device 112 may provide a response 120 to the server 104 that indicates that a medical condition is present. The server 104 may then transmit an activation signal to each member user device of a medical team in order to activate a team of medical professionals to handle the medical condition. In reference to FIG. 4, a first activation signal 134 may be sent by server 104 to member user device 132 and a second activation signal 144 may be sent by server 104 to member user device 142. Each activation signal may separately require a team member to confirm receipt of their respective activation signal in order to indicate that they are fulfilling their role in preparing to treat patient with the identified medical condition.

Before transmitting activation signal 134 and activation signal 144, server 104 may first identify member user device 132 and member user device 142 as being associated with viewing device 112 as part of a medical team responsible for handling the medical condition represented in medical image data 108. For instance, one set of medical providers may be identified by a hospital administrator as being responsible for handling a STEMI while a second set of medical providers may be identified by a hospital administrator as being responsible for handling an aortic dissection. In some examples, the member user devices may each be smartphones associated with users that are members of one or more medical teams responsible for particular medical conditions.

In further examples, each member user device may be sent additional information from server 104 as part of an activation signal. For instance, each member user device may separately have a software application that allows for viewing of the medical image data 108 before the end of a timeout period. Additionally, the activation signals may also turn on mobile device functionality of member user devices as well or instead. For instance, GPS functionality may be activated in order to cause the member user devices to transmit location information to server 104. This location information may be used, for instance, to determine how long to make particular medical image data available for viewing.

In additional examples, activation signals may be sent to other types of computing devices as well or instead. In particular, the server 104 may send an activation signal to initialize a piece of medical equipment at a hospital (e.g., cath lab equipment). Additionally, the activation signal may include instructions to initialize the equipment based on the medical condition identified from medical image data 108 and confirmed by viewing device 112. The activation signal may also require the equipment to provide closed-loop acknowledgement when activation has occurred.

Next, FIG. 5 illustrates additional communication between a server and member user devices of a medical team, in accordance with example embodiments. More specifically, member user device 132 may transmit a confirmation signal 136 to server 104 after a user of member user device 132 enters input data confirming receipt of the activation signal 134. In some examples, confirmation signal 136 may only be sent after confirming the identity of the user of member user device 132 (e.g., using a finger print or a different identifier). Similarly, member user device 142 may transmit a separate confirmation signal 146 to server 104 after a user of member user device 142 enters input data confirming receipt of the activation signal 144.

The server 104 may monitor received confirmation signals to ensure that each team member of a medical team confirms that they are activated and preparing to treat the patient. If a team member doesn't confirm receipt, the server 104 may take remedial action such as resending the activation signal or sending an activation signal to a member user device associated with a backup team member. In additional examples, application alerts, phone calls, and/or paging functionality may be used by the server 104 in order to ensure that closed-loop acknowledgment is received from each member user device. In this manner, the image sharing application may not only ensure that medical image data is received by the right doctor and a clinical decision is made for a patient, but also may close the loop to activate an entire team of medical professionals to prepare to do a medical procedure on the patient.

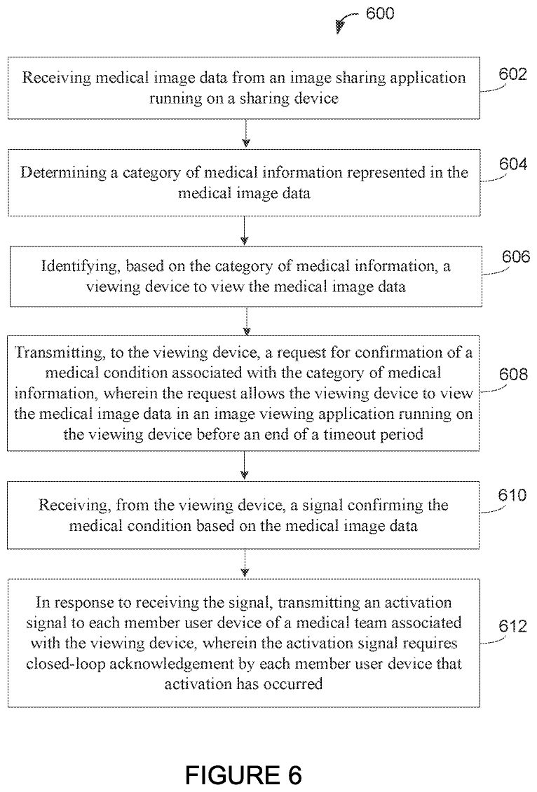

FIG. 6 is a block diagram of an example method 600. Method 600 shown in FIG. 6 presents an embodiment of a method that could be used or implemented by server 104, for example, or by components of such a computing device, or more generally by any of a variety of computing devices. Method 600 may involve communication with one or more smartphones running particular software applications, and/or with different types of mobile devices, such as wearable devices. Method 600 can include one or more operations, functions, or actions as illustrated by one or more of blocks 602-612. Although the blocks are illustrated in a sequential order, these blocks can also be performed in parallel, and/or in a different order than those described herein. Also, the various blocks can be combined into fewer blocks, divided into additional blocks, and/or removed based upon the desired implementation.

Additional example methods may be carried out by a sharing device in communication with a remote server. Yet further example methods may be carried out by a viewing device in communication with a remote server. In addition, for the method 600 and other processes and methods disclosed herein, the block diagram shows functionality and operation of one possible implementation of present embodiments. In this regard, each block can represent a module, a segment, or a portion of program code, which includes one or more instructions executable by a processor or computing device for implementing specific logical functions or steps in the process. The program code can be stored on any type of computer-readable medium, for example, such as a storage device including a disk or hard drive. The computer-readable medium can include non-transitory computer-readable medium, for example, such as computer-readable media that stores data for short periods of time like register memory, processor cache and random access memory (RAM). The computer-readable medium can also include non-transitory media, such as secondary or persistent long term storage, like read only memory (ROM), optical or magnetic disks, compact-disc read only memory (CD-ROM), for example. The computer-readable medium can also be any other volatile or non-volatile storage systems. The computer-readable medium can be considered a computer-readable storage medium, for example, or a tangible storage device.

In addition, for the method 600 and other processes and methods disclosed herein, each block in FIG. 6 can represent circuitry that is wired to perform the specific logical functions in the process.

At block 602, method 600 includes receiving medical image data from an image sharing application running on a sharing device. The sharing device may be a smartphone or a different type of user device with a camera. The image sharing application may be a software application that allows for the capture of medical image data and the transmission of such data to a remote server without permanently storing the medical image data on the user device. The medical image data may be a single frame, multiple images, a portion of video data, or a different type of image data. The particular portion of medical image data shared may be customized by a user of the sharing device via the image sharing application before the medical image data is received by the remote server.

At block 604, method 600 includes determining a category of medical information represented in the medical image data. More specifically, the medical image data may be processed to determine whether the medical image data falls into one of a plurality of predefined categories corresponding to different types of medical imaging technology (e.g., MRIs, CT scans, X-rays, EKGs). In further examples, the sharing device may provide information identifying the category of medical information represented in the medical image data. In additional examples, a server may determine multiple levels of categorization of the medical image data. For instance, in addition to determining what type of imaging technology generated a medical image, the image may be further processed to determine what part of the body it relates to or what type of clinical decision is needed.

At block 606, method 600 includes identifying a viewing device to view the medical image data. More specifically, a server may identify a viewing device based at least on the category of medical information. Additionally, the server may reference a scheduling application to determine available doctors. For instance, the server may determine to send an EKG image to the smartphone of an on-call cardiologist at a hospital that is proximate to a patient being treated by EMS in the field. In some examples, the sharing device may be responsible for identifying at least the hospital from which to select viewing devices. In further examples, the sharing device may provide further information regarding what type of doctor is needed or what type of clinical decision is needed to further assist with identification of an appropriate viewing device.

At block 608, method 600 includes transmitting, to the viewing device, a request for confirmation of a medical condition associated with the category of medical information. For instance, the request may be for confirmation of the presence of STEMI when the medical image data falls within the category of EKG image data. In some examples, the relevant medical condition to be diagnosed may be determined by the server by processing the medical image data. In other examples, the medical condition may be identified by the sharing device. The request allows the viewing device to view the medical image data in an image viewing application running on the viewing device before an end of a timeout period. The image viewing application is a software application that may display the shared medical image data without saving the medical image data to the viewing device. In some examples, the image sharing application and the image viewing application may both be part of a single application. In other examples, a user device may only have sharing or viewing capabilities, but not both.

At block 610, method 600 includes receiving, from the viewing device, a signal confirming the medical condition based on the medical image data. The signal may require that the user of the viewing device input identifying information, such as a finger print. In some examples, the signal may indicate a yes/no response from the user of the viewing device. In other examples, the signal may indicate a medical condition from a list of three or more possibilities. In further examples, the signal may also indicate that a clinical decision cannot be made based on the shared medical image data.

At block 612, method 600 includes, in response to receiving the signal, transmitting an activation signal to each member user device of a medical team associated with the viewing device. Each member user device may be identified as belonging to a team member on a medical team of doctors on call to handle the medical condition confirmed based on the medical image data. The activation signal sent to each member user device may require closed-loop acknowledgement by each member user device that activation has occurred. If each team member does not confirm receipt of the activation signal, the server may send reminders or take other remedial actions to ensure that the team is activated to handle the patient. In further examples, one or more activation signals may be sent to other types of computing devices as well or instead of user devices. The other types of computing devices may also be required to confirm activation via closed-loop communication.

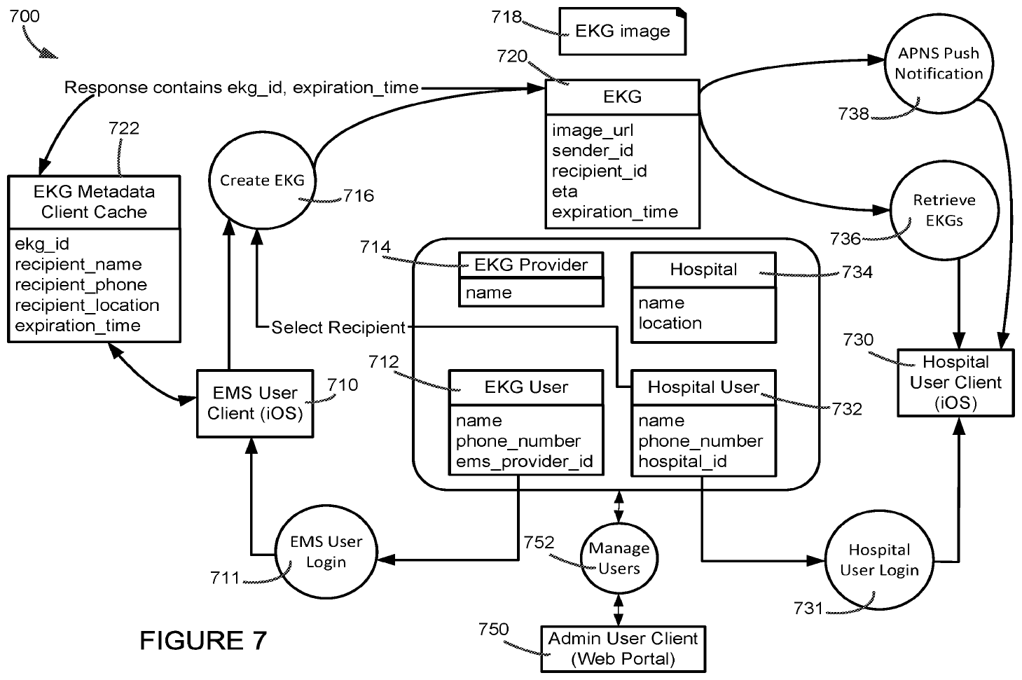

FIG. 7 is a data flow diagram, in accordance with example embodiments. More specifically, the diagram includes components of an example system that facilitates transmission of an EKG image from the field. In particular, the system 700 includes EMS User Client 710, Hospital User Client 730, and Admin User Client 750. EMS User Client 710 is a software application that may be run on a mobile device (e.g., camera phone) of an EMS worker. Hospital User Client 730 is a software application that may be run on a mobile device of a hospital user (e.g., a cardiologist). Admin User Client 750 is a software application that may be run through a web portal by a system administrator. In some examples. EMS User Client 710, Hospital User Client 730, and/or Admin User Client 750 may include common functionality (e.g., a single application may allow for both sending and viewing of EKG images).

EMS User 712 is a particular user associated with EMS Provider 714. In order to access EMS User Client 710, EMS User 712 may input login credentials, shown here as EMS User Login 711. For example, EMS User 712 may sign in using a phone number and a password set by an admin user who access Manage Users 752 through Admin User Client 750. When an authorized user such as EMS User 712 signs in, the system may generate an access token and send it in a response to the user. EMS User Client 710 caches this token for subsequent application programming interface (API) requests. The token is destroyed on sign out, or a subsequent sign in.

In response to user input from EMS User 712, EMS User Client 710 may capture and send EKG images to a server. This process is shown in FIG. 7 as Create EKG 716. In particular, EMS User Client 710 may capture EKG image 718, which is a photograph of an EKG machine readout. EMS User Client 710 may also create a digital EKG object 720 or EKG artifact. The EKG object 720 may include metadata associated with EKG image 718. In particular, the EKG object 720 may include a link image_url to view EKG image 718. The EKG object 720 may additionally include sender_id identifying EMS User 712 and/or EMS Provider 714. The EKG object 720 may further include recipient_id identifying Hospital User 732 and/or Hospital 734, the EKG object 720 may also include an estimated time of arrival (ETA) indicating a predicted clock time or a predicted amount of time (e.g., a number of minutes) for the EMS user to reach the hospital with a patient. The EKG object 720 may further include an expiration_time indicating a time at which the EKG object 720 and associated EKG image 718 will no longer be available (e.g., 120 minutes from image capture). In further examples, EKG object 720 may include more or fewer components of metadata than those specifically listed in this example.

EKG image 718 may potentially contain protected health information (PHI). In some examples, EKG image 718 is never stored to disk on the device of EMS User 712. Additionally in some examples, after EMS User 712 sends EKG image 718 to a server, it can never be viewed by EMS User 712 again. The transmission of EKG image 718 from EMS User Client 710 to a server may be secured using HTTPS.

In some examples, EMS User Client 710 may cache some metadata in client cache 722 about the recipient of the EKG object 720 at send time. The client cache 722 may be stored on a device of the EMS User 712 so that the metadata may be displayed within a ‘Sent EKGs’ list on the device. The metadata may include ekg_id, an identifier that has been assigned to the EKG at the server; recipient_name, the name of Hospital User 732; recipient_phone, the phone number of Hospital User 732; recipient_location, the location of Hospital 734; and expiration_time, the time at which the EKG object 720 will expire and no longer be accessible from the server. More or fewer pieces of information may be stored in client cache 722. Such information may include an identification number of the sender, an identification number of the recipient, a ‘created at’ date of the EKG image from the backend system, a name of the destination hospital, and an ETA of the EMS to the destination hospital (which may be calculated at send time).

EKG object 720 including EKG image 718 may be received from EMS User Client 710 at a remote server (not shown). On the server, before EKG image 718 is stored to disk, it may be encrypted. For instance, advanced encryption standard (AES) 256 cipher block chaining (CBC) may be used with the initialization vector (IV) randomized for each image. On the server, the encrypted image may be stored in a directory that is not publicly accessible. Access may only be exposed to the image through an authenticated endpoint that returns the decrypted image as a base64-encoded string, so that only the authorized user can receive it. EKG image 718 may only be stored on the server for a predetermined time (e.g., 120 minutes). In some examples, after the predetermined time expires. EKG image 718 may be deleted from the filesystem and database along with the rest of the EKG metadata stored in EKG object 720. In other examples, such data may be retained for quality control, federal guideline adherence, and/or other reasons.

Only the recipient specified by the sender (in this case, Hospital User 732) is able to download the EKG image from the server using Hospital User Client 730. Hospital User 732 may be able to login to Hospital User Client 730 at Hospital User Login 731. For example, Hospital User 732 may sign in using a phone number and a password set by an admin user. Once logged in. Hospital User 732 may be able to view EKG image 718 and associated metadata in EKG object 720, including the ETA of the EMS to the hospital. In some examples, the image is never stored on the recipient's phone; no client-side caching occurs so that each time the recipient wants to view the image, it must be downloaded again from the server. In particular, EKG objects in Hospital User Client 730 are only stored in memory; they are not cached to disk.

The Hospital User Client 730 may actively send a request to the server for available EKGs for Hospital User 732 at Retrieve EKGs 736. More specifically, every time the application becomes active, it may perform a fresh authenticated GET operation to the server to obtain all unexpired EKGs and corresponding metadata whose recipient is the logged-in hospital user. In further examples, the server may send a push notification 738 to Hospital User Client 730 each time an EKG object is received that is directed to a hospital user that logged in to Hospital User Client 730. An example push payload may include an ekg_id that identifies the received EKG object as well as a generic message such as “You have received a new EKG.”

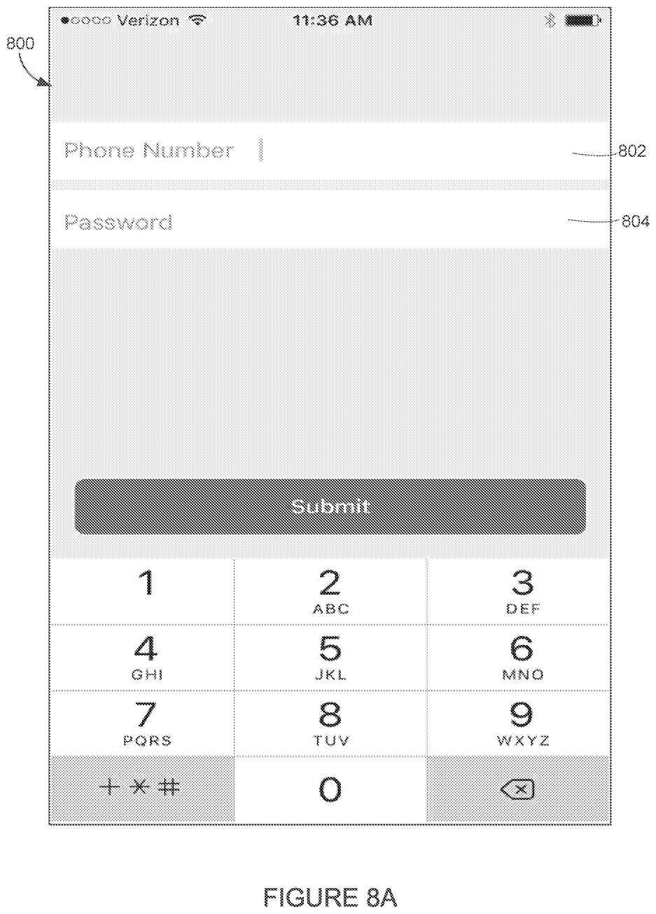

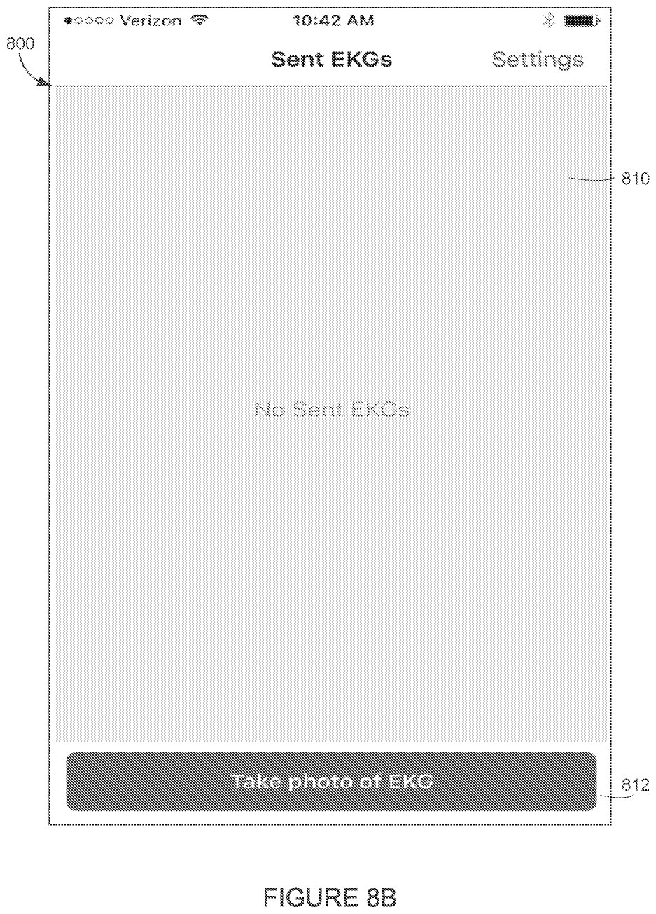

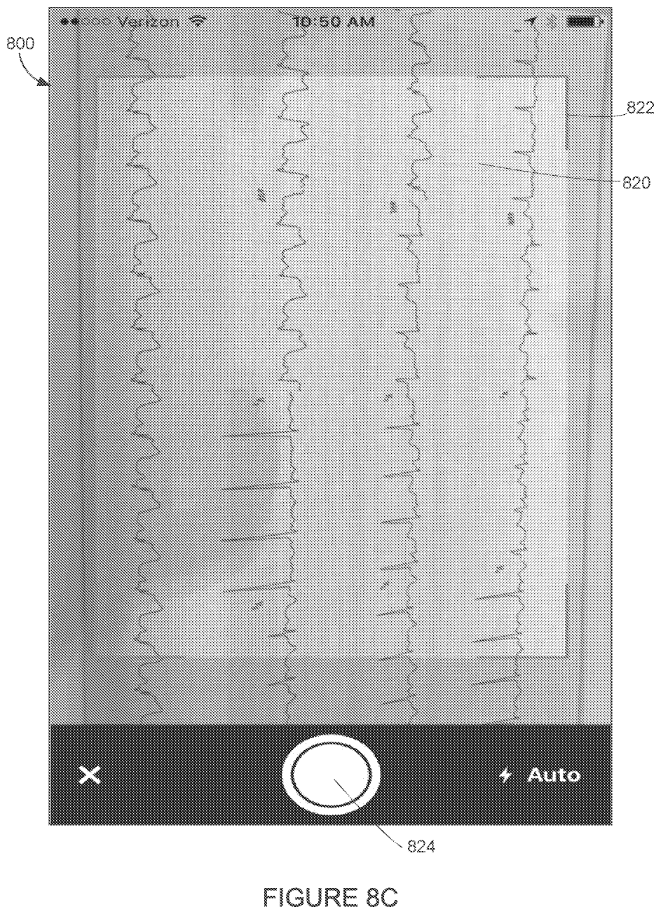

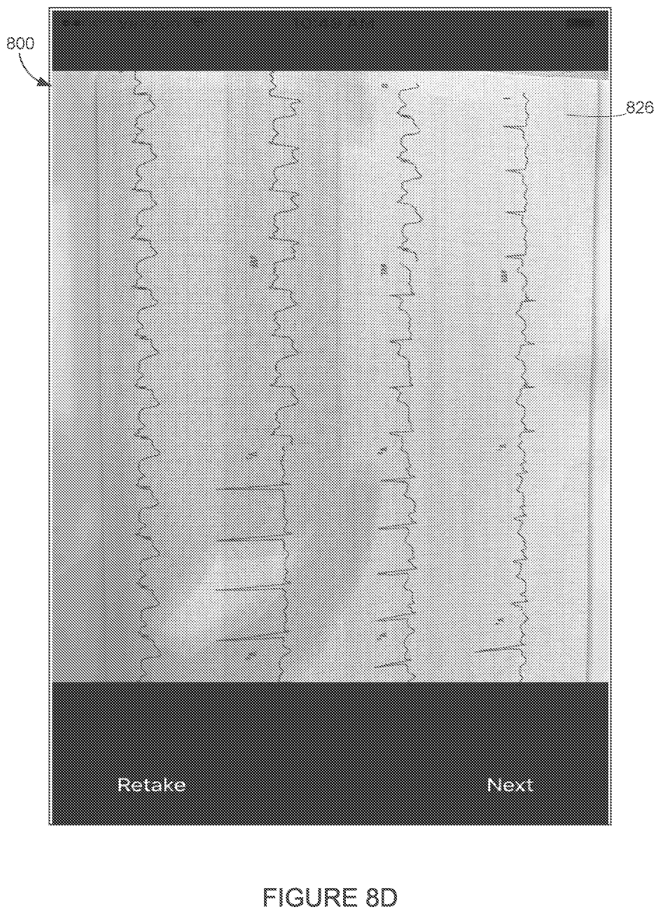

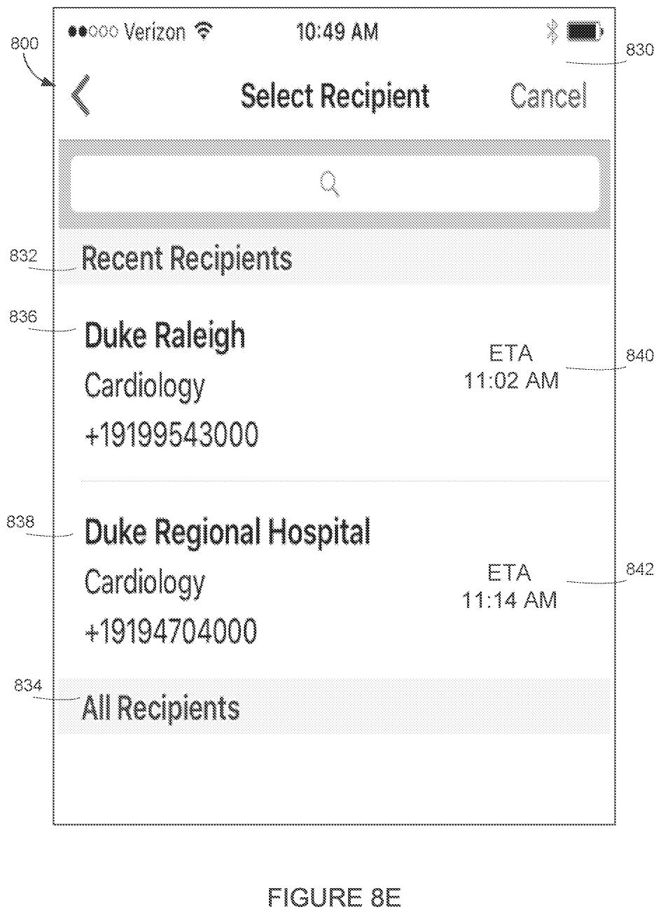

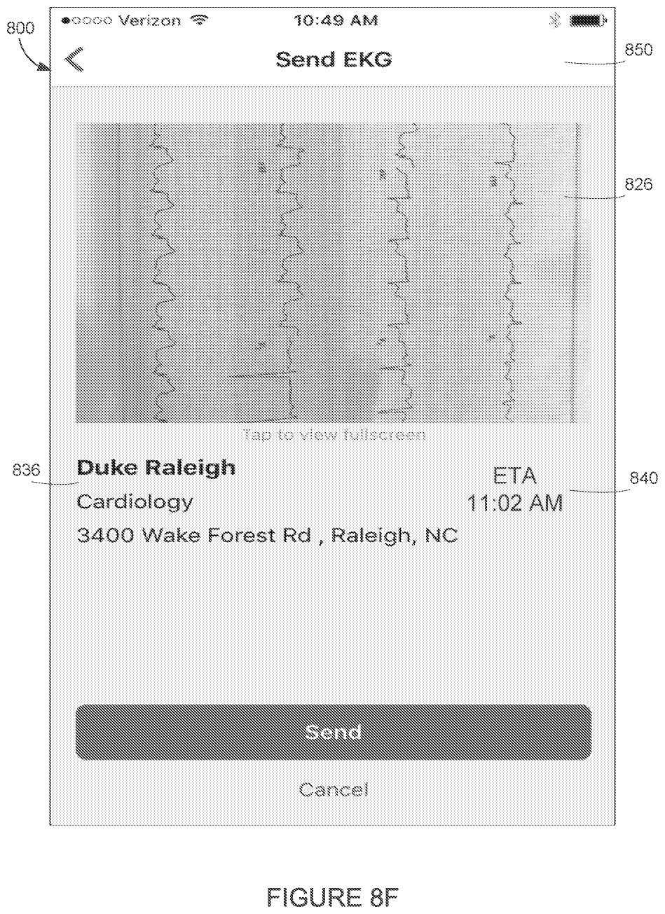

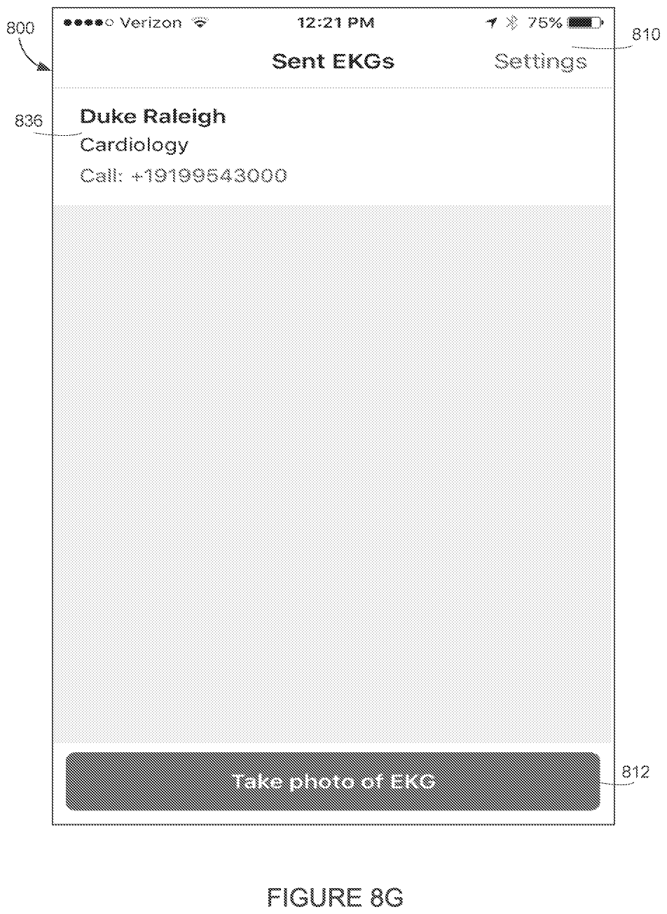

FIGS. 8A-8G illustrate an example series of application screenshots from a sender device. The screenshots may be displayed to a user such as an EMS worker who may use the application in order to transmit an EKG image to a hospital user such as a cardiologist. In some examples, the sender device is a mobile phone with a camera. In other examples, the sender device is a tablet device, a wearable device, or a different type of mobile device with a camera.

FIG. 8A shows an example login screen in order to access the application. More specifically, a camera phone 800 may run the application in response to a user input. Before the user is allowed to use the application to take and send an EKG image, the user may be required to enter phone number 802 and password 804. Other types of login credentials may also be required before allowing an EMS user to access the application.

FIG. 8B shows an example Sent EKGs screen that may be displayed to an EMS user who has logged in to the application. In particular, Sent EKGs list 810 may include certain metadata about recently transmitted EKG images by the logged-in user. Notably, the list 810 may not include the EKG images. Accordingly, in order to protect sensitive patient information, the user may only be able to view a captured EKG image for verification before transmitting the EKG image. After logging out and logging back in, previously sent EKG images may not be accessible from within the application. In this case, the list 810 indicates no sent EKGs. In some examples, the metadata describing sent EKGs may be removed from the list 810 after a predetermined time period (e.g., a day or a week). The application also displays a button 812 to switch to a screen in which the user may capture an EKG image.

FIG. 8C shows an example EKG image capture screen that may be displayed by the application. In particular, when an EMS user is triaging a patient, the EMS user may access an EKG machine that provides EKG printout 820 which shows electric activity of the patient's heart. The application may overlay a bounding box 822 on EKG printout 820 to show what portion of the EKG printout 820 will be captured by the camera when the user presses button 824. The user may first be requested to enable camera access to the application before the screen shown in FIG. 8C is reached or before button 824 is made active. Notably, a captured EKG image may be stored locally by the application without saving to permanent storage on camera phone 800. Accordingly. EKG images captured by the application will be inaccessible by a user of the phone outside of the application.

The size of the bounding box 822 may be determined by the application. In some examples, the bounding box 822 may be set in order to capture a predetermined amount of voltage measurements while excluding surrounding parts of the EKG printout 820. In particular, if the EKG printout 820 contains a patient name, date of birth, or other personal health information, the application may determine the size of the bounding box 822 to exclude this information. In some examples, the application may have a number of different EKG templates corresponding to printouts from different models of EKG images. Each template may have a different shape and size for the bounding box 822. The application may then identify the type of EKG machine, and then determine the shape and size of the bounding box based on the stored template for that type of EKG machine.

FIG. 8D shows an example EKG image captured within the application. In particular, EKG image 826 may be captured from within the application. The area of EKG image 826 may correspond to the area of the bounding box displayed by the application on top of an EKG printout. The user may be able to view EKG image 826 within the application and retake the image if desired.

FIG. 8E shows an example recipient selection screen within the application. After a user captures an EKG image and approves of the captured image, the application may then display a list of possible recipients for the image. In particular, the application may display a Select Recipient screen 830. As shown here, recent recipients 832 to whom the logged-in user recently transmitted an EKG image be separated out and displayed before all other recipients 834. The application may locate nearby hospitals with an activated cath lab to display within the list, such as Duke Raleigh 836 and Duke Regional Hospital 838. The user may select a hospital from the list in order to route a EKG image to an appropriate recipient at the hospital.

In some examples, the application may also determine and list an ETA to each hospital, shown here as ETA 840 for Duke Raleigh 836 and ETA 842 for Duke Regional Hospital 838. The application may communicate with a global positioning system (GPS) on the mobile phone 800 in order to locate nearby hospitals, and times and/or distances to the hospitals. In order to compute ETAs, a user may be prompted to enable location access on the phone to the application. The hospitals may be sorted so that the hospital with the earliest ETA is listed first. The ETA of the selected hospital may be stored as metadata associated with an EKG image. Notably, the ETA may be stored only as a clock time or a quantity of time (e.g., a number of minutes) without location information that include PHI, such as an address or a zip code.

In further examples, the application may also include a scheduling feature that maintains schedules for all cardiologists in the system. The scheduling feature may allow the specific user who is “on call” to automatically roll over to the next scheduled provider after a pre-specified date and time. For example, a first cardiologist at a hospital may be on call from 7/1/2017 at 8:00 A.M. until 7/8/2017 at 7:59 A.M.; a second cardiologist at the hospital may be scheduled to start taking calls at 8:00 A.M. on 7/8/2017. The scheduling feature may allow the provider identified as the “on-call” provider to automatically update at a pre-specified date and time (e.g., as inputted by an administrator or scheduler). The roll over process may be a passive process that automatically changes the user who is notified of and may view incoming EKG images. In further examples, a confirmation message may be provided to the outgoing cardiologist (e.g., “You have completed your call!”) and/or the incoming cardiologist (e.g., “You are now on call, please swipe right to confirm or press decline to indicate that you are not scheduled to be on call.”).

The application may also display a phone number for each hospital in the select recipient list 830. The phone number may allow an EMS user to call a cardiologist user at the hospital directly from the application, either before or after transmitting an EKG image.

FIG. 8F shows an example send confirmation screen. In particular, the application may display the Send EKG window 850 to allow an EMS user to confirm sending of EKG image 826 to a recipient at selected hospital 836. The ETA 840 may be included as metadata with EKG image 826 as part of a digital EKG object. In some examples, after the user clicks to send the image, the EKG image 826 may be deleted from memory and no longer accessible to the user. In other examples, after a message confirming receipt of the image by the hospital is received, the EKG image 826 may then be deleted from memory. The EKG and metadata may be transmitted over HTTPS and encrypted at rest on the server, and only stored for a predetermined time period (e.g., 60 minutes) on the server.

FIG. 8G shows an example sent EKGs window with a sent EKG entry. In particular, Sent EKGs list 810 now displays an entry corresponding to the EKG image just sent to Duke Raleigh 836. The last entry on the list may serve as a reminder to the EMS user of what hospital has been provided the EKG image to remind the EMS user where to drive the patient. The entry may include a phone number which allows the user to contact the target hospital. In some examples, the application may host a phone number that will directly contact the target hospital. In further examples, an EMS user may be provided with a phone number to directly call a cardiologist at the target hospital from the application. Notably, the EKG image itself is not displayed or accessible from within the Sent EKGs list 810. Accordingly, the application minimizes access to the EKG image in an effort to protect the patient's PHI. The application also displays a button 812 to take another photo of an EKG to allow the user to restart the process to capture and transmit a different EKG image to a recipient at a hospital.

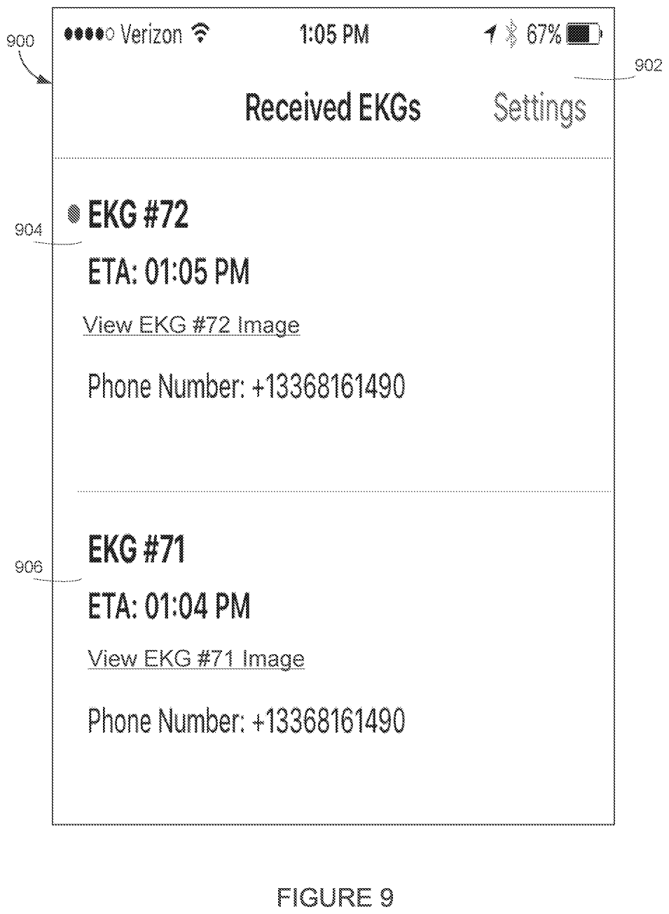

FIG. 9 shows an example application screenshot of a viewer device. More specifically, device 900 may be a computing device used by a cardiologist at a hospital to access an application to view EKG images transmitted from the field. Device 900 may be a mobile phone or some other type of mobile or stationary device. The viewer application may display a received EKGs list 902 with recently uploaded EKG images for the logged-in user. In this example, the list 902 includes EKG 904 and EKG 906. In some examples, every time the application launches or becomes active, it makes a fresh request to retrieve all unexpired EKGs that have sent to the logged-in user.

The received EKGs list 902 includes links to view the images for EKG 904 and EKG 906. In some examples, the EKG images are never stored to disk on the recipient's phone. No client-side caching of the image occurs. Each time the recipient wants to view the image, it must be downloaded again from server, and the EKG images can only be viewed from within the application by clicking the links within the received EKGs list 902.

In some examples, when a hospital user views one of the EKGs, the application caches the identifier of the viewed EKG so that the application can update an ‘unviewed’ indicator. No other data about the EKGs is stored in the client, and this cached identifier is removed as soon as the corresponding EKG no longer shows up in the list 902. In FIG. 9, a circular dot indicates that EKG 904 has been read or addressed by the hospital user, while EKG 906 has not been viewed or addressed.

The received EKGs list 902 also includes ETAs corresponding to each of the received EKG images. The ETAs may be provided by a server which parses the metadata associated with received EKG images. In some examples, the ETAs may be displayed only as a fixed clock time or fixed amount of time. In other examples, the ETAs may be updated each time a user logs in to view an EKG image by receiving updated ETA information from a sender's device.

The received EKGs list 902 also includes phone numbers corresponding to each of the received EKG images. The application provides the hospital user with the option to directly call the EMS user from within the application.

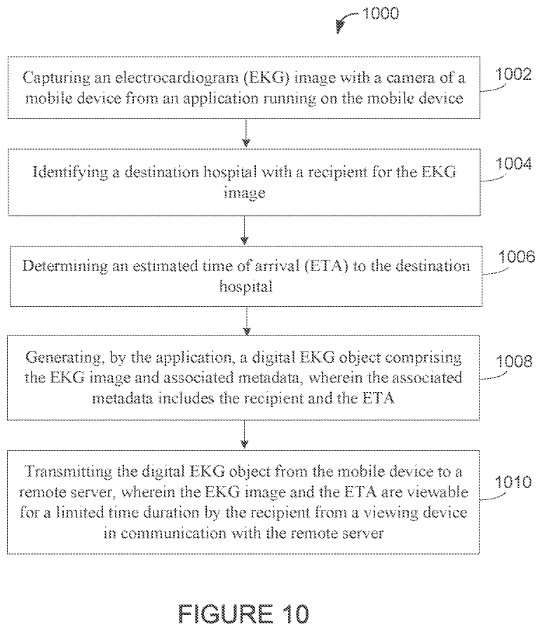

FIG. 10 is a block diagram of an example method 1000. Method 1000 shown in FIG. 10 presents an embodiment of a method that could be used or implemented by computing device running EMS User Client 710 of FIG. 7, for example, or by components of such a computing device, or more generally by any of a variety of computing devices. Method 1000 may be carried out by a camera phone, or a different type of mobile device with a camera, such as a wearable device. Method 1000 can include one or more operations, functions, or actions as illustrated by one or more of blocks 1002-1010. Although the blocks are illustrated in a sequential order, these blocks can also be performed in parallel, and/or in a different order than those described herein. Also, the various blocks can be combined into fewer blocks, divided into additional blocks, and/or removed based upon the desired implementation.

Additional example methods may be carried out by a remote server in communication with a sender device and a viewer device. Yet further example methods may be carried out by a viewer device in communication with a remote server. In addition, for the method 1000 and other processes and methods disclosed herein, the block diagram shows functionality and operation of one possible implementation of present embodiments. In this regard, each block can represent a module, a segment, or a portion of program code, which includes one or more instructions executable by a processor or computing device for implementing specific logical functions or steps in the process. The program code can be stored on any type of computer-readable medium, for example, such as a storage device including a disk or hard drive. The computer-readable medium can include non-transitory computer-readable medium, for example, such as computer-readable media that stores data for short periods of time like register memory, processor cache and random access memory (RAM). The computer-readable medium can also include non-transitory media, such as secondary or persistent long term storage, like read only memory (ROM), optical or magnetic disks, compact-disc read only memory (CD-ROM), for example. The computer-readable medium can also be any other volatile or non-volatile storage systems. The computer-readable medium can be considered a computer-readable storage medium, for example, or a tangible storage device.

In addition, for the method 1000 and other processes and methods disclosed herein, each block in FIG. 10 can represent circuitry that is wired to perform the specific logical functions in the process.

At block 1002, method 1000 includes capturing an EKG image with a camera of a mobile device, such as a mobile phone. The EKG image may be captured from within an application running on the mobile device. The EKG image may not be stored to the mobile device. In some examples, the application may display a bounding box on the mobile device to assist a user in capturing the EKG image. In further examples, the application may determine dimensions of the bounding box to exclude one or more pieces of PHI from the EKG image. In additional examples, the application may determine dimensions of the bounding box based on one or more previously stored EKG image templates.

At block 1004, method 1000 may further include identifying a destination hospital with a recipient for the EKG image. In some examples, the destination hospital may be selected by a user from a list of nearby hospitals with activated cath labs. In further examples, a hospital may only be displayed in the list if the hospital has an associated recipient (e.g., a cardiologist) that is logged into a viewing application that allows for viewing received EKG images. In further examples, a recipient at the hospital may automatically be selected by the application after the user selects the destination hospital. In particular, a scheduling feature may identify the current “on-call” cardiologist who is currently scheduled to receive and view EKG images at the destination hospital.

At block 1006, method 1000 may additionally include determining an ETA to the destination hospital. The ETA may be determined by communicating with a GPS system on the mobile device. In some examples, the ETA may be determined as a clock time or an amount of time without any location information associated with a location of the mobile device. In additional examples, ETAs to each of several nearby hospitals may be determined. The hospitals may then be ordered within a list by ETA to enable a selection of a hospital by the user.

At block 1008, method 1000 may further include generating a digital EKG object by the application. The digital EKG object may include the EKG image and associated metadata. The associated metadata may include the recipient for the EKG image and the ETA. In further examples, different pieces of metadata may be stored with the EKG image as well or instead. In additional examples, the EKG image may be sent without any associated metadata.

At block 1010, method 1000 may additionally include transmitting the digital EKG object from the mobile device to a remote server. The EKG image and the ETA may be viewable for a limited time duration (e.g., 120 minutes) by the recipient from a viewing device in communication with the remote server. In some examples, the limited time duration may be a predetermined fixed time interval starting from a time when the EKG image was captured. In additional examples, the limited time duration may be determined by the application based on the ETA. For instance, the limited time duration may be set to the ETA plus an additional 10 minutes to minimize the amount of time that the remote server keeps ownership of the data.

In further examples, after transmitting the EKG image, certain metadata associated with the EKG image may be stored in a list of recently transmitted EKGs, including the recipient and the time sent. In some examples, the EKG image may not be viewable within the list of transmitted EKGs. Additionally, the EKG image may not be viewable at all by the sending device after the image has been transmitted, or after confirmation is received that the image was successfully transmitted to the remote server. More specifically, the digital EKG object may be erased from a local cache on the mobile device after transmitting the digital EKG object or in response to receiving an input signal at the mobile device indicating to transmit the digital EKG object.

In a further example system, post-image processing may be done using the EKG images and associated cardiologist decisions whether or not to activate a cath lab. In particular, one or more machine learning processes may be used to train a system to learn when a cardiologist is likely to activate a cath lab for a given EKG image based on past cardiologist decisions for similar images. A machine learning model (e.g., a neural net) may then be trained to evaluate an EKG image and automatically determine whether or not cath lab activation is needed for the patient. In such systems, an EKG image may be input into the machine earning model and a cath lab may automatically be activated or not based on output from the machine learning model.

In other example systems, other images, video, text, or types of data may be transmitted by a system that uses the same or similar components as described herein.

The particular arrangements shown in the Figures should not be viewed as limiting. It should be understood that other embodiments may include more or less of each element shown in a given Figure. Further, some of the illustrated elements may be combined or omitted. Yet further, an illustrative embodiment may include elements that are not illustrated in the Figures.

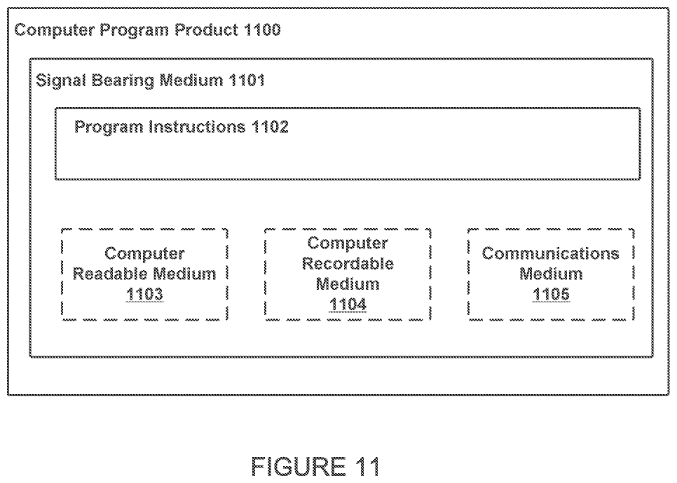

In some embodiments, the disclosed methods can be implemented as computer program instructions encoded on a non-transitory computer-readable storage media in a machine-readable format, or on other non-transitory media or articles of manufacture. FIG. 11 is a schematic illustrating a conceptual partial view of an example computer program product 1100 that includes a computer program for executing a computer process on a computing device, arranged according to at least some embodiments presented herein.

In one embodiment, the example computer program product 1100 is provided using a signal bearing medium 1101. The signal bearing medium 1101 can include one or more programming instructions 1102 that, when executed by one or more processors can provide functionality or portions of the functionality described above with respect to FIGS. 1-10. In some examples, the signal bearing medium 1101 can encompass a computer-readable medium 1103, such as, but not limited to, a hard disk drive, a Compact Disc (CD), a Digital Video Disk (DVD), a digital tape, memory, etc. In some implementations, the signal bearing medium 1101 can encompass a computer recordable medium 1104, such as, but not limited to, memory, read/write (R/W) CDs, R/W DVDs, etc. In some implementations, the signal bearing medium 1101 can encompass a communications medium 1105, such as, but not limited to, a digital and/or an analog communication medium (e.g., a fiber optic cable, a waveguide, a wired communications link, a wireless communication link, etc.). Thus, for example, the signal bearing medium 1101 can be conveyed by a wireless form of the communications medium 1105 (e.g., a wireless communications medium conforming to the Institute of Electrical and Electronic Engineers (IEEE) 802.11 standard or other transmission protocol).

The one or more programming instructions 1102 can be, for example, computer executable and/or logic implemented instructions. In some examples, a computing device can be configured to provide various operations, functions, or actions in response to the programming instructions 1102 conveyed to the computing device by one or more of the computer-readable medium 1103, the computer recordable medium 1104, and/or the communications medium 1105.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements can be omitted altogether according to the desired results. Further, many of the elements that are described are functional entities that can be implemented as discrete or distributed components or in conjunction with other components, in any suitable combination and location.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

1. A method comprising:
receiving medical image data from an image sharing application running on a sharing device;
determining a category of medical information represented in the medical image data;
identifying, based on the category of medical information, a viewing device to view the medical image data;
transmitting, to the viewing device, a request for confirmation of a medical condition associated with the category of medical information, wherein the request allows the viewing device to view the medical image data in an image viewing application running on the viewing device before an end of a timeout period;
receiving, from the viewing device, a signal confirming the medical condition based on the medical image data; and
in response to receiving the signal, transmitting an activation signal to each member user device of a medical team associated with the viewing device, wherein the activation signal requires closed-loop acknowledgement by each member user device that activation has occurred.

2. The method of claim 1, further comprising:
determining the timeout period based at least on the category of medical information represented in the medical image data; and
erasing the medical image data from a database at the end of the timeout period.

3. The method of claim 1, further comprising:
determining, based on Global Positioning System (GPS) data from the sharing device, an estimated time of arrival (ETA) of the sharing device at a hospital; and
determining the end of the timeout period based on the ETA of the sharing device.

4. The method of claim 1, further comprising:
determining, based on GPS data from the viewing device, an ETA of the viewing device at a hospital; and
determining the end of the timeout period based on the ETA of the viewing device.

5. The method of claim 1, further comprising:
providing an indication of the timeout period for display on the sharing device;
receiving input data from the sharing device indicating an adjustment to the timeout period; and
adjusting the timeout period based on the input data.

6. The method of claim 1, further comprising:
providing an indication of the timeout period for display on the viewing device;
receiving input data from the viewing device indicating an adjustment to the timeout period; and
adjusting the timeout period based on the input data.

7. The method of claim 1, further comprising:
providing an indication of the timeout period for display on both the sharing device and the viewing device; and
receiving input data from both the sharing device and the viewing device indicating confirmation or adjustment of the timeout period as part of a handshake process, wherein the medical image data is stored in a database subsequent to receiving confirmation of the timeout period from each of the sharing device and viewing device during the handshake process.

8. The method of claim 1, further comprising providing for display of a toggleable option on the sharing device, wherein the toggleable option indicates whether to determine timeout periods for subsequently captured images or to permanently store the subsequently captured images in a database.

9. The method of claim 1, wherein the category of medical information is selected from a plurality of predefined categories comprising at least magnetic resonance imaging (MRI) data, electrocardiogram (EKG) data, X-ray data, and computerized tomography (CT) scan data.

10. The method of claim 1, further comprising validating the medical image data based on the category of medical information before transmitting the request.

11. The method of claim 1, wherein the request to the viewing device comprises a request to verify that the medical image data is interpretable.

12. The method of claim 1, further comprising determining one or more portions of the medical image data to block from view by the viewing device.

13. The method of claim 12, where the one or more portions of the medical image data to block from view by the viewing device are determined based on the category of medical information represented in the medical image data.

14. The method of claim 12, further comprising receiving input data from the sharing device indicating the one or more portions of the medical image data to block from view by the viewing device.

15. The method of claim 12, further comprising:
subsequent to determining the one or more portions of the medical image data to block from view by the viewing device, providing indications of the one or more portions of the medical image data for display on the sharing device; and
receiving input data from the sharing device indicating one or more adjustments to locations of the one or more portions of the medical image data; and
storing the locations of the one or more portions of the medical image data to block from view by the viewing device in a database.

16. The method of claim 12, wherein the one or more portions of the medical image data correspond to one or more pieces of personal health information (PHI) identified in the medical image data.

17. The method of claim 1, wherein the medical image data comprises a plurality of frames from video data captured by a video camera on the sharing device, wherein the plurality of frames are identified based on input data from the sharing device.

18. The method of claim 1, wherein the medical image data comprises a plurality of frames from video data captured by a video camera on the sharing device, wherein the plurality of frames are identified based on the category of medical information represented in the medical image data.

19. A non-transitory computer readable medium having stored therein instructions
executable by one or more processors to cause the one or more processors to perform functions comprising:
receiving medical image data from an image sharing application running on a sharing device;
determining a category of medical information represented in the medical image data;
identifying, based on the category of medical information, a viewing device to view the medical image data;
transmitting, to the viewing device, a request for confirmation of a medical condition associated with the category of medical information, wherein the request allows the viewing device to view the medical image data in an image viewing application running on the viewing device before an end of a timeout period;
receiving, from the viewing device, a signal confirming the medical condition based on the medical image data; and
in response to receiving the signal, transmitting an activation signal to each member user device of a medical team associated with the viewing device, wherein the activation signal requires closed-loop acknowledgement by each member user device that activation has occurred.

20. A system comprising:
one or more processors; and
a non-transitory computer readable medium having stored therein instructions executable by the one or more processors to cause the one or more processors to perform functions comprising:
receiving medical image data from an image sharing application running on a sharing device;
determining a category of medical information represented in the medical image data;
identifying, based on the category of medical information, a viewing device to view the medical image data;
transmitting, to the viewing device, a request for confirmation of a medical condition associated with the category of medical information, wherein the request allows the viewing device to view the medical image data in an image viewing application running on the viewing device before an end of a timeout period;
receiving, from the viewing device, a signal confirming the medical condition based on the medical image data; and
in response to receiving the signal, transmitting an activation signal to each member user device of a medical team associated with the viewing device, wherein the activation signal requires closed-loop acknowledgement by each member user device that activation has occurred.

21-40. (canceled)
